(19) United States
(12) Patent Application Publication
(10) Pub. No.: US 2006/0008428 A1
(43) Pub. Date: Jan. 12, 2006

(54) Method of promoting the penetration of a cosmetic active and composition therefore

(75) Inventors: Nathalie Seyler, Maisons-Alfort (FR); Carole Guiramand, Jouy En Josas (FR); Christele Ribaud, Nogent S/Marne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/153,380

(22) Filed: Jun. 16, 2005

Related U.S. Application Data
(60) Provisional application No. 60/621,804, filed on Oct. 26, 2004.

(30) Foreign Application Priority Data
Jun. 16, 2004 (FR) 0406539

(51) Int. Cl. A61K 8/18

(52) U.S. Cl. 424/59

(57) ABSTRACT

The invention relates to a method of promoting the penetration of a cosmetic active, wherein a composition in the form of a water-in-oil emulsion is applied topically, said emulsion comprising a fatty phase with a polarity ba greater than or equal to 0.1 and comprising a mixture of at least two oils, and in that the application of said emulsion is made simultaneously, with an interval or sequentially in time with the application of the cosmetic active.

### REFERENCE TO PRIOR APPLICATIONS

This application claims priority to U.S. provisional application 60/621,804 filed Oct. 26, 2004, and to French patent application 0406539 filed Jun. 16, 2004, both incorporated herein by reference.

### FIELD OF THE INVENTION

The present invention relates to a method of promoting the penetration of at least one cosmetic active into keratin material such as the hair, mucous membranes, lips, the skin and/or scalp, etc. and hence of improving its activity by increasing the rate and/or amount of active traversing the horny layer to reach its site of action. It also relates to compositions that can be used for improving the penetration of a cosmetic or dermatological active.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The skin is composed of two compartments, one on the surface, the epidermis, and one lower down, the dermis, which interact. Natural human epidermis is composed principally of three types of cell, these being the keratinocytes, the vast majority, the melanocytes, and the Langerhans' cells. Each of these cell types contributes, through its specific functions, to the essential role played by the skin in the body, particularly the role of protecting the body from external aggressive influences, which is referred to as the “barrier function”.

The epidermis is conventionally divided into a basal layer of keratinocytes, which constitutes the germinative layer of the epidermis, a so-called prickle cell layer, consisting of a number of layers of polyhedral cells arranged on the germinative layers, from one to three layers known as granular layers, which consist of flattened cells containing distinct cytoplasmic inclusions, the keratohyalin granules, and, finally, the horny layer (or stratum corneum), which consists of a collection of layers of keratinocytes at the final stage of their differentiation, referred to as corneocytes.

The dermis provides the epidermis with a solid support. It is also its nutritional element. It consists principally of fibroblasts and of an extracellular matrix composed primarily of collagen, elastin and a substance called ground substance. These components are synthesized by the fibroblasts. Also present therein are leukocytes, mastocytes or else tissue macrophages. Finally, the dermis is traversed by blood vessels and nerve fibres.

Cohesion between the epidermis and the dermis is ensured by the dermal-epidermal junction.

The topical administration of active compounds very often comes up against a problem of penetration of the horny layer or stratum corneum such that said compounds are unable to reach the deeper layers of the skin, the epidermis and dermis.

A variety of solutions have been proposed for improving skin permeability for formulations containing active agents on underlying layers of the epidermis or of the dermis. However, there still exists a need for cosmetic formulations which are well tolerated and effective for combating this barrier effect of the stratum corneum with respect to the diffusion of actives.

U.S. 2003/0017176 describes compositions of water-in-oil emulsion type which exhibit a low viscosity and contain a high level of water, and necessarily include silicone surfactants.

EP 1342463 describes compositions for the lips, such as sticks, which allow the bitter taste of the sunscreen bisethyloxyphenylmethoxyphenyltriazine to be masked; it is known that it is desirable for UV screens to remain on the skin's surface.

U.S. 2003/0108579 describes anhydrous compositions comprising at least one polyol ether, one oil and one wax, which is solid at 37° C.

U.S. Pat. No. 5,489,429 describes water-in-oil emulsions for handcare, comprising a mixed ester of isostearic acid and succinic acid with glycerol, plus at least 10% by weight of polyol.

### SUMMARY OF THE INVENTION

Unexpectedly the applicant has found that the treatment or pre-treatment of the skin's surface with a cosmetic composition exhibiting a polar oily phase comprising at least two oils promotes the skin penetration of actives.

### DETAILED DESCRIPTION OF THE PREFERRED EMBODIMENTS

The present invention provides a method of promoting the penetration of a cosmetic active, wherein a composition in the form of a water-in-oil emulsion is applied topically, said emulsion comprising a fatty phase with a polarity δa greater than or equal to 0.1 and comprising a mixture of at least two oils, and in that the application of said emulsion is made simultaneously, with an interval or sequentially in time with the application of the cosmetic active.

The polarity may be described by the Hansen solubility parameter δa; throughout this text it is expressed in J^{1/2} cm^{-3/2}, unless specified otherwise. This is because this parameter, for a given constituent, characterizes the energy corresponding to the polar (δp) and hydrogen-bond-type (δh) interactions existing between the molecules of this constituent.
δₐ=\sqrt{δₚ²+δₕ²}

As recalled in the work “Properties of Polymers” by D. W. Van Krevelen, 3rd edition (Elsevier, 1990), page 200 et seq., the solubility of a compound in a given solvent is to a large extent determined by its chemical structure. Apolar oils have a δa value of 0. Polar oils have a δa value other than 0, i.e. greater than 0.

The composition in water-in-oil (W/O) emulsion form is applied to the skin and/or scalp before or at the same time as the active principle whose penetration is to be improved, so as to increase the amount of active reaching its site of action and/or the rate of penetration of the active, and hence its bioavailability.

A cosmetic active in the present text denotes a compound or mixture of compounds, in purified form or in complex form, especially mineral or plant-based, exhibiting an intrinsic activity in vitro or in vivo, and capable of formulation within a cosmetic product. A “cosmetic product” is, in particular, any substance or preparation intended to be brought into contact with the various surface parts of the human body (epidermis, body-hair and head-hair system, nails, lips and external genital organs) or with the teeth and the buccal mucosae for the purpose, exclusively or principally, of cleaning them, of fragrancing them, of modifying their appearance and/or of correcting body odours and/or of protecting them or of keeping them in good condition (Cosmetics Directive 76/768/EEC, amended).

The method according to the invention will obviously also be appropriate for promoting the penetration of two or more actives.

According to one embodiment of the invention the W/O emulsion comprises a mixture of at least three oils, the fatty phase exhibiting a final δa value of greater than or equal to 0.1.

Preferably the final polarity of the fatty phase is characterized by a δa value of greater than or equal to 0.2, in particular greater than or equal to 0.4, such as 0.5, 0.6, 0.7, 0.8, 1, 2, 3, 4, etc.

Generally the final polarity of the fatty phase of the emulsion corresponds to a δa value of less than or equal to 8, in particular less than or equal to 5, or even less than or equal to 4.

The fatty phase of the emulsion may comprise a mixture of apolar oils and polar oils, the respective amounts of which will be adapted by the skilled person to give a fatty phase whose δa values are in accordance with the invention.

For example, the fatty phase of the emulsion preferably comprises from 20% to 80%, relative to the total weight of the fatty phase, in particular from 40% to 75%, of polar oils possessing δa values of between 2 and 11. According to one embodiment of the invention the polar oils present in the fatty phase have individual δa values of 3 to 8, in particualr greater than 3.9.

According to one of the advantageous embodiments of the invention the oils of the fatty phase comprise at least one oil whose viscosity is less than or equal to 10 cst.

Preferably the compositions according to the invention are liquid or fluid at 37° C., or even at ambient temperature, i.e. approximately 25° C.

Included among the oils present in the composition mention may be made of oils of silicone type and/or of synthetic-oil type or synthetic-ester type and/or of vegetable-oil type with a high triglyceride content; it is possible to select hydrocarbon oils, silicone oils and/or fluoro oils. In particular the emulsion will comprise at least one polar oil selected from vegetable oils having a high triglyceride content, synthetic oils and/or synthetic-ester oils and silicone oils.

An “oil” is any non-aqueous medium which is liquid at ambient temperature (25° C.) and atmospheric pressure (760 mm Hg) and is physiologically acceptable.

These oils may be hydrocarbon oils and/or silicone oils and/or fluoro oils. They may be animal, plant, mineral or synthetic in origin. A “hydrocarbon oil” is any oil comprising on a majority basis atoms of carbon and of hydrogen, and optionally ester, ether, fluoro, carboxylic acid and/or alcohol groups. Moreover, the oils used may be volatile and/or non-volatile. A volatile oil is any oil capable of undergoing evaporation at ambient temperature from a substrate to which it has been applied; in other words, an oil having a vapour tension, measurable at 25° C. and 1 atmosphere, of, for example, greater than 0 Pa, in particular ranging from 10⁻³ to 300 mm Hg (0.13 Pa to 40 000 Pa). As volatile oils mention may be made in particular of volatile silicone oils, such as linear or cyclic volatile silicones. Mention may also be made of volatile hydrocarbon oils such as isoparaffins, and volatile fluoro oils.

Among the oils which can be used in the composition of the invention, some are polar and others are apolar (i.e. non-polar).

The polar oils comprise in their chemical structure at least one nonionic polar group, and preferably at least two ionic or nonionic polar groups, such as the following groups:

COOH;

mono- or disubstituted OH (primary or secondary);

PO₄;

NHR; NR₁R₂, R₁ and R₂ optionally forming a ring and representing a C₁ to C₂₀ linear or branched alkyl or alkoxy radical, or 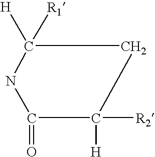
where R₁′ and R₂′ may represent hydrogen or a C₁ to C₂₀ linear or branched alkyl or alkoxy chain. Apolar oils have a δa value of 0. In particular the apolar oils according to the invention may be selected in particular from:

linear or branched hydrocarbons of mineral or synthetic origin, such as volatile or non-volatile liquid paraffins and derivatives thereof; vaseline oil; liquid lanolin; polydecenes; hydrogenated polyisobutenes such as Parleam® oil; squalane; hydrogenated isoparaffin; isohexadecane; isododecane;

and mixtures thereof.

The polar oils have a δa value other than 0, i.e. greater than 0. In particular the polar oils used in the composition of the invention may be selected from:

oils of plant origin, hydrocarbon oils having a high triglyceride content, composed of esters of fatty acids and glycerol wherein the fatty acids may have various chain lengths from C₂ to C₂₄, it being possible for said chains to be linear or branched, saturated or unsaturated. As oils of plant origin mention may be made in particular of jojoba oil, wheat germ oil, maize oil, sunflower oil, karite butter oil, castor oil, sweet almond oil, macadamia oil, apricot oil, soya oil, cotton oil, alfalfa oil, poppy oil, pumpkin oil, sesame oil, marrow oil, colza oil, avocado oil, hazelnut oil, grape seed oil, blackcurrant seed oil, evening primrose oil, millet oil, barley oil, quinoa oil, olive oil, rye oil, safflower oil, candlenut oil, passionflower oil, musk rose oil or coriander oil; or else caprylic/capric triglycerides such as those sold by Stearineries Dubois or those sold under the names Miglyol 810, 812 and 818 by Dynamit Nobel;

synthetic oils or synthetic esters of formula R₅COOR₆ in which R₅ represents the residue of a linear or branched fatty acid containing 1 to 40 carbon atoms and R₆ represents a hydrocarbon chain, in particular a branched hydrocarbon chain, containing 1 to 40 carbon atoms, with the proviso that R₅+R₆ is ≧10, such as, for example, Purcellin oil (cetostearyl octanoate), isononyl isononanoate, C₁₂ to C₁₅ alcohol benzoate, isopropyl myristate, 2-ethylhexyl palmitate, isostearyl isostearate, isopropyl isostearate, alcohol or polyalcohol octanoates, decanoates or ricinoleates; hydroxy esters such as isostearyl lactate and diisostearyl malate; 2-octyldodecyl stearate, 2-octyldodecyl erucate, isostearyl isostearate; hydroxy esters such as isostearyl lactate, octyl hydroxystearate, octyldodecyl hydroxystearate, diisostearyl malate, triisocetyl citrate, heptanoates, polyol esters, such as propylene glycol dioctanoate, neopentyl glycol diheptanoate and diethylene glycol diisononanoate; pentaerythritol esters such as pentaerythrityl tetraisostearate; lipophilic derivatives of amino acids, such as isopropyl lauroyl sarcosinate (INCI name: isopropyl lauroyl sarcosinate), sold under the name Eldew SL 205 by Ajinomoto;

synthetic ethers having 10 to 40 carbon atoms;

C₈ to C₂₆ fatty alcohols, such as oleyl alcohol, isostearyl alcohol and octyldodecanol;

silicone oils, such as linear or cyclic, volatile or non-volatile polydimethylsiloxanes (PDMS) which are liquid at ambient temperature; phenyl silicones such as phenyltrimethicones, phenyldimethicones, phenyl-trimethylsiloxydiphenylsiloxanes, diphenyldimethicones, diphenylmethyldiphenyltrisiloxanes, 2-phenylethyl trimethylsiloxysilicates, polymethylphenylsiloxanes and alkyl silicones;

mixtures thereof.

However, according to one of the aspects of the invention, the fatty alcohols which can be used do not include unsaturated C₁₈ fatty alcohols.

Among the silicones, preference will be given to selecting dimethicones. Among synthetic esters or oils, preference will be given to synthetic esters or oils of formula R₅COOR₆ in which R₅ represents the residue of a linear or branched fatty acid containing 1 to 40 carbon atoms and R₆ represents a hydrocarbon chain, in particular a branched hydrocarbon chain, containing 1 to 40 carbon atoms, with the proviso that R₅+R₆ is > or =10. Preference will be given in particular to isononyl isononanoate and isopropyl lauroyl sarcosinate. Among the vegetable oils with a high triglyceride content, preference will be given to selecting apricot kernel oil.

The composition can be prepared conventionally by the skilled person, in view of this disclosure, it being possible for the amount of fatty phase in the emulsion to vary, in particular from 2.5% to 50% by weight relative to the total weight of the composition, especially from 4% to 40%.

The compositions of the invention may further comprise conventional cosmetic adjuvants selected in particular from fats, organic solvents, thickeners, softeners, opacifiers, stabilizers, emollients, antifoams, moisturizers, perfumes, preservatives, polymers, fillers, sequestrants, propellants, alkalifying or acidifying agents or any other ingredient commonly used in cosmetology.

In particular the O/W composition preferably includes an emulsifier (or surfactant), in an amount for example from 0.1% to 40% by weight relative to the total weight of the composition, in particular from 0.5% to 20%.

Water-in-oil emulsions may be stabilized by various surfactants: in particular, alkyl derivatives of polyglycerol, alkylated polyethylene glycols, alkylated sorbitan derivatives, metal salts of fatty acids, silicone surfactants, and, preferably, by oligomers or polymers derived from polyolefins. In one of the embodiments the surfactant is selected from the group consisting of alkyl derivatives of polyglycerol, alkylated polyethylene glycols, alkylated sorbitan derivatives, metal salts of fatty acids, and oligomers or polymers derived from polyolefins. In particular, silicone surfactants may be essentially absent from the composition intended to enhance penetration according to the invention. Mention may be made for example, as emulsifiers, of dimethicone copolyols such as the mixture of cyclomethicone and dimethicone copolyol sold under the name DC 5225 C by Dow Corning, and alkyldimethicone copolyols such as the laurylmethicone copolyol sold under the name Dow Corning 5200 Formulation Aid by Dow Corning and the cetyldimethicone copolyol sold under the name Abil EM 90R by Goldschmidt, or the poly-4-glyceryl isostearate/cetyldimethicone copolyol/hexyl laurate mixture sold under the name Abil WE 09 by Goldschmidt.

It is also possible to add one or more co-emulsifiers. Advantageously the co-emulsifier may be selected from the group consisting of polyol alkyl esters. As polyol alkyl esters mention may be made in particular of glycerol esters and/or sorbitan esters and, for example, polyglycerol isostearate, such as the product sold under the name Isolan GI 34 by Goldschmidt, sorbitan isostearate, such as the product sold under the name Arlacel 987 by ICI, glycerol sorbitan isostearate, such as the product sold under the name Arlacel 986 by ICI, and mixtures thereof.

As W/O emulsions surfactant it is also possible to use a crosslinked elastomeric solid organopolysiloxane comprising at least one alkoxylated group, such as those obtained according to the procedure of Examples 3, 4 and 8 of document U.S. Pat. No. 5,412,004 and the examples of document U.S. Pat. No. 5,811,487, particularly the product of Example 3 (synthesis example) of U.S. Pat. No. 5,412,004, and of the kind sold under the reference KSG 21 by Shin Etsu.

Oligomers and polymers derived from polyolefins and useful as emulsifiers in the composition of the invention are composed of a polyolefinic apolar moiety and at least one polar moiety. They may exhibit a block- or comb-type structure. The polyolefinic apolar moiety comprises at least 40 carbon atoms and preferably from 60 to 700 carbon atoms. This apolar moiety may be selected from polyolefins such as the oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins are hydrogenated or non-hydrogenated.

Furthermore, the polyolefin-derived oligomers or polymers used in the composition of the invention comprise at least one polar moiety. This polar moiety endows the polyolefin derivatives with amphiphilic properties. Thus these oligomers or polymers lower the interfacial tension (water/oil, in other words between aqueous phase and oily phase) by at least 10 mN/m when they are present at a concentration of 0.01% by weight relative to the total weight of the oily phase.

The polar moiety of the oligomeric or polymeric emulsifiers of the invention may be anionic, cationic, nonionic, zwitterionic or amphoteric. It is composed for example of polyalkylene glycols or of polyalkyleneimines, or else of carboxylic or dicarboxylic acids, of their anhydrides or of derivatives thereof, and mixtures thereof. Oligomeric or polymeric emulsifiers having a polar carboxylic acid moiety may be obtained, for example, from the reaction of a polyolefin with at least one carboxylic acid or anhydride selected from the group consisting of maleic acid, maleic anhydride, fumaric acid, itaconic acid, citraconic acid, mesaconic acid and aconitic acid. Preferably the polar moiety is composed of succinic acid or anhydride, their ester or amide derivatives, the corresponding alkali metal, alkaline earth metal or organic ion salts, or else of polyoxyethylene.

The emulsifiers derived from polyoxyethylene may be selected for example from polyisoprene-polyoxyethylene diblock polymers, poly(ethylene-co-propylene)-polyoxyethylene polymers and mixtures thereof. These polymers are described in the publication by Allgaier, Poppe, Willner, Richter (Macromolecules, 1997, vol. 30, pp. 1582-6).

Emulsifiers derived from succinic acid or anhydride may be selected in particular from the polyolefin derivatives of succinic acid or anhydride that are described in U.S. Pat. No. 4,234,435, U.S. Pat. No. 4,708,753, U.S. Pat. No. 5,129,972, U.S. Pat. No. 4,931,110, GB-A-2,156,799 and U.S. Pat. No. 4,919,179, incorporated here for reference. The polyolefin moiety may be composed, for example, of hydrogenated or non-hydrogenated polyisobutylene with a molecular weight ranging from 400 to 5000. In the resultant polyisobutylene having a succinic end group, the succinic moiety may be esterified, amidated or in salt form; that is, it may be modified with alcohols, amines, alkanolamines or polyols, or else may be in the form of alkali metal or alkaline earth metal salts, ammonium salts or else organic-based salts such as diethanolamine and triethanolamine salts. Polyolefins having an esterified or amidated succinic end group are products of reaction of (a) a polyolefin having a succinic end group and (b) an amine or an alcohol, to form an amide or an ester. The term “amine” used here embraces all types of amines, including alkanolamines. The amines in question may be, for example, primary, secondary or tertiary monoamines, it being possible for these amines to be aliphatic, cycloaliphatic, aromatic, heterocyclic and saturated or unsaturated. Furthermore, the alcohols may be mono- or polyalcohols. Monoalcohols include primary, secondary or tertiary aliphatic alcohols and phenols. The polyalcohols may be selected, for example, from aliphatic, cycloaliphatic, aromatic and heterocyclic polyalcohols. The polyolefins having a modified (esterified or amidated) succinic end group and the process for preparing them are described in particular in document U.S. Pat. No. 4,708,753, which is incorporated here for reference.

Oligomers and polymers derived from polyolefins suitable for the invention are advantageously polyolefins having a succinic end group; mention may be made in particular of polyisobutylenes having a modified succinic end group, such as the products sold under the names L2724, L2721 and Lubrizol 5603® by Lubrizol, Hostacerin® PIB by Clariant and Chemcinnate 2000® by Chemron.

Another example of a polymeric surfactant which can be used in the invention is the product of the reaction of maleic anhydride with polyisobutylene, such as the product sold under the name Glissopal SA by BASF. The amount of emulsifying oligomer(s) or polymer(s) in the composition of the invention may range, for example, from 0.1% to 10% by weight of active substance, preferably from 0.5% to 5% by weight and more preferably from 1% to 3% by weight relative to the total weight of the composition. It is possible to use one or more polyolefin-derived oligomers or polymers. According to one preferred embodiment of the invention the polyolefin-derived oligomers or polymers are the only emulsifiers used in the composition according to the invention. However it is possible optionally to add other amphiphilic agents which are commonly used in water-in-oil emulsions, such as conventional ionic, nonionic, amphoteric or zwitterionic surfactants, amphiphilic oligomers or polymers, or amphiphilic organic or inorganic particles.

According to one embodiment of the invention, the compositions intended for promoting penetration are essentially free of isostearic acid and/or the surfactants are not co-emulsified with succinic acid.

One of the subjects of the invention is a method of promoting the penetration of a cosmetic active as defined above, which comprises the following steps:

(a) the water-in-oil emulsion is applied to an area of the skin and/or scalp,

(b) the emulsion is left in contact with the skin and/or scalp for a time greater than or equal to 2 minutes,

(c) the cosmetic active is applied to the same area of the skin and/or scalp as that of step (a).

According to one of the embodiments of the invention, steps (a) and (c) are carried out sequentially in time, and the cosmetic active is present in a cosmetic composition comprising a physiologically acceptable medium.

According to another embodiment of the invention, steps (a) and (c) are carried out simultaneously, and the cosmetic active is present in the composition in the form of a water-in-oil emulsion of step (a).

By “physiologically acceptable” is meant a medium which is compatible with the skin, mucosae, lips and/or epidermal derivatives, in particular the nails or the hair; preferably the medium will be cosmetically acceptable, i.e., it exhibits an appearance, feel, odour and, where appropriate, taste which are pleasant for the user.

The compositions of step (a) include, optionally, a cosmetically or dermatologically acceptable vehicle. Where step (c) is carried out with a time interval relative to step (a), the composition of step (c) may be in any of the formulating forms that are normally used for topical application, and in particular in the form of an aqueous, aqueous-alcoholic or oily solution, an oil-in-water or water-in-oil or multiple emulsion, an aqueous or oily gel, a liquid, pasty or solid anhydrous product, a spherule-mediated dispersion of oil in an aqueous phase, it being possible for these spherules to be polymer nanoparticles such as nanospheres and nanocapsules or, more preferably, lipid vesicles of ionic and/or nonionic type.

The composition which can be used in steps (a) and/or (c) may be more or less fluid and may have the appearance of a white or coloured cream, an ointment, a milk, a lotion, a serum, a paste or a mousse. It may optionally be applied to the skin in an aerosol form. It may also be present in a solid form, and for example in stick form. It may be used as a care product, such as a cleansing product, as a makeup product or else as a simple deodorant product.

The composition of the invention may also comprise the adjuvants such as those which are common in the fields of cosmetology and dermatology, such as hydrophilic or lipophilic gelling agents, hydrophilic or lipophilic actives, preservatives, antioxidants, solvents, perfumes, fillers, screening agents, pigments, chelating agents, odour absorbers and colorants. The amounts of these various adjuvants are those which are commonly used in the fields in question, and amount for example to from 0.01% to 20% of the total weight of the composition. These adjuvants, depending on their nature, may be introduced into the fatty phase, into the aqueous phase, into the lipid vesicles and/or into the nanoparticles. The compositions according to the invention may comprise pigments or nanopigments (average size of the primary particles generally between 5 nm and 100 nm, preferably from 10 nm to 50 nm) of metal oxides coated or not, such as titanium, iron, zinc, zirconium or cerium oxide which are known photoprotective UV agent. Such nanopigments of metal oxides are for instance disclosed in patent applications EP 518772 and EP518773.

When UV screening agents are present in the composition, their molecular weight is preferably greater than or equal to 500, and/or they are combined with structures such as microcapsules in order to limit their passage through the skin. According to one particular embodiment the composition contains no bisethyloxyphenolmethoxyphenyltriazine, or, if this screening agent is present, it is combined with at least one other UV screening agent.

The contact time of step (b) will be adapted to provide the degree and rate of penetration appropriate to the active principles and to the cosmetic activity that are targeted in a selected embodiment.

For example, the W/O emulsion will be applied to the whole or part of the face and neck in the form of a pretreatment lotion or mask for a duration of 2 to 20 minutes, in particular approximately 5 to 10 minutes, although this duration may be prolonged without disadvantage, for instance until one or several hours. Where steps (a) and (c) are separated by a time interval, the composition, containing in particular a moisturizing, calming or anti-ageing active, is subsequently applied to the face and neck and is left in place for at least 5 minutes, optionally for at least one day or night.

The method can also be a slimming method, and the W/O emulsion according to the invention is applied on the body area where fat deposit are to be reduced, such as legs, arms, stomach or bottom of the face, in a simultaneously, separately or sequentially with a lipolytic or slimming agent.

According to another aspect, the method is intended to slow down the loss of hair and/or to increase the diameter and/or density of the hair shaft, or for lessening canities: the W/O emulsion according to the invention is applied in the form of a mask, cream or hair lotion for pretreatment of the scalp; subsequently the composition containing the active is applied, in the form for example of a shampoo, which is subsequently rinsed off, or of a lotion which is intended to remain in contact with the scalp for at least one day or even until the next shampooing.

The cosmetic actives suitable for the implementation of the invention include actives whose activity is developed in layers deeper than the stratum corneum, and which therefore must not remain on the surface of the skin, in order to have optimum efficacy.

Mention may be made in particular of moisturizers, desquamating agents, anti-seborrhoeic agents, anti-ageing agents, depigmenting or anti-pigmenting agents, pigmenting or pro-pigmenting agents, muscle relaxants or dermal decontractants, calmatives, lipolytic or slimming agents, microcirculation promoters and hair-loss reducers and/or hair-growth promoters.

The anti-ageing agents may be selected, for example, from free-radical scavengers, anti-glycation agents, NO synthase inhibitors, agents which stimulate the synthesis of dermal or epidermal macromolecules and/or prevent their degradation, agents which stimulate fibroblast or keratinocyte proliferation and/or prevent keratinocyte differentiation, keratolytic agents, vitamins, anti-elastase and anti-collagenase agents, fatty acid derivatives, trace elements, algal and planktonic extracts, enzymes, coenzymes, flavonoids and muscle relaxants.

Desquamating Agents and Moisturizers

A “desquamating agent” is any compound capable of acting

either directly on desquamation, by promoting exfoliation, such as β-hydroxy acids, particularly salicylic acid and its derivatives (including 5-n-octanoylsalicylic acid, a-hydroxy acids, such as glycolic, citric, lactic, tartaric, malic or mandelic acid; urea; gentisic acid; oligofucoses; cinnamic acid; extract of Saphora japonica; resveratrol and certain jasmonic acid derivatives;

or on the enzymes involved in desquamation or in corneodesmosome degradation, glycosidases, stratum corneum chymotryptic enzyme (SCCE) or even other proteases (trypsin, chymotrypsin-like). Mention may also be made of chelating agents for mineral salts: EDTA; N-acyl-N,N′,N′-ethylenediaminetriacetic acid; aminosulphonic compounds and particularly (N-2-hydroxyethylpiperazine-N-2-ethane)sulphonic acid (HEPES); 2-oxothiazolidine-4-carboxylic acid derivatives (procysteine); alpha-amino acid derivatives of glycine type (as described in EP-0 852 949, and also the sodium methylglycine diacetate sold by BASF under the trade name Trilon M); honey; and sugar derivatives such as O-octanoyl-6-D-maltose and N-acetylglucosamine.

A “moisturizer” means, in particular, a compound acting on the barrier function, in order to maintain the hydration of the stratum corneum. Mention may be made of ceramides, sphingoid-based compounds, lecithins, glycosphingolipids, phopholipids, cholesterol and its derivatives, phytosterols (stigmasterol, β-sitosterol, campesterol), essential fatty acids, 1,2-diacylglycerol, 4-chromanone, and pentacyclic triterpenes such as ursolic acid.

These compounds may represent, for example, from 0.001% to 30%, and preferably from 0.01% to 20%, of the total weight of the composition according to the invention.

The composition according to the present invention comprising the abovementioned desquamating agents and moisturizers is advantageously intended for preventing or treating the drying-out of the skin, and in particular xeroses.

Depigmenting, Anti-Pigmenting or Pro-Pigmenting Agent

As whitening or depigmenting agents mention may be made, for example, of kojic acid and its derivatives; hydroquinone and its derivatives such as arbutin and its esters; vitamin C and its derivatives such as magnesium ascorbylphosphate; salts such as calcium D-pantetheinesulphonate; ellagic acid and its derivatives; rucinol; linoleic acid and its derivatives; plant extracts, and particularly extracts of liquorice, of mulberry or of skullcap and of Bacopa monnieri, without this list being limitative; glutathione and its precursors; cysteine and its precursors; the aminophenol-derived compounds described in document WO-A-99/10318, such as, in particular, N-ethyloxycarbonyl-4-aminophenol, N-ethyloxycarbonyl-O-ethyloxycarbonyl-4-aminophenol, N-cholesteryloxy-carbonyl-4-aminophenol, N-ethylaminocarbonyl-4-aminophenol; and mixtures of these compounds.

As a pro-pigmenting agent mention may be made of extract of burnet (Sanguisorba officinalis), sold by Maruzen, and extracts of chrysanthemum (Chrysanthemum morifolium).

The composition according to the present invention comprising the abovementioned depigmenting agents is advantageously intended for the prevention or treatment of hyperpigmentation, in particular of pigmentary spots associated with ageing of the skin.

For its part, the composition comprising the aforementioned pro-pigmenting agents is intended in particular for the treatment of canities, or to promote pigmentation of the skin.

Anti-Glycation Agent

An “anti-glycation agent” is a compound which prevents and/or lessens the glycation of the proteins of the skin, particularly the proteins of the dermis such as collagen.

Examples of anti-glycation agents are plant extracts from the family of the Ericaceae, such as an extract of blueberry (Vaccinium angustifolium); ergothioneine and its derivatives; and hydroxystilbenes and their derivatives, such as resveratrol and 3,3′,5,5′-tetrahydroxystilbene. These anti-glycation agents are described in Applications FR 2 802 425, FR 2 810 548, FR 2 796 278 and FR 2 802 420, respectively.

The composition according to the invention comprising an anti-glycation agent as defined above may advantageously be used for preventing or treating the signs of skin ageing, and in particular for preventing or treating loss of tone and/or elasticity in the skin.

NO Synthase Inhibitor

Examples of NO synthase inhibitors that are suitable for use in the present invention include, in particular, a plant extract of the species Vitis vinifera, which is sold in particular by Euromed under the name Leucocyanidines de raisins extra, or else by Indena under the name Leucoselect®, or, finally, by Hansen under the name Extrait de marc de raisin; a plant extract of the species Olea europaea, which is preferably obtained from olive leaves and is sold in particular by Vinyals in the form of a dry extract, or by Biologia & Technologia under the trade name Eurol BT; and an extract of a plant of the species Gingko biloba, which is preferably a dry aqueous extract of this plant, sold by Beaufour under the trade name Ginkgo biloba extrait standard.

The composition according to the invention comprising an NO synthase inhibitor as defined above may advantageously be used for preventing or treating signs of skin ageing and/or sensitive skins.

Anti-Seborrhoeic Agent

When the composition according to the invention comprises an anti-seborrhoeic agent such as a 5α-reductase inhibitor, said agent may be selected in particular from:
retinoids, and especially retinol;
sulphur and sulphur derivatives;
zinc salts such as the lactate, gluconate, pidolate, carboxylate, salicylate and/or cysteate of zinc;
vitamin B6 or pyridoxine;
the mixture of capryloyl glycine, sarcosine and extract of Cinnamomum zeylanicum sold in particular by SEPPIC under the trade name Sepicontrol A5®;
an extract of Laminaria saccharina, sold in particular by SECMA under the trade name Phlorogine®;
an extract of Spirea ulmaria sold in particular by Silab under the trade name Sebonormine®;
plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;
an extract of Serenoa repens sold in particular by Euromed;
extracts of plants of the genus Silybum;
plant extracts containing sapogenins, and in particular the hecogenin- or diosgenin-rich extracts of Dioscorea plants, and
extracts of Eugenia caryophyllata containing eugenol and eugenyl glucoside.

The anti-seborrhoeic agent represents, for example, from 0.001% to 10%, and preferably from 0.01% to 5%, of the total weight of the composition according to the invention. When said composition includes such a compound, it is particularly well suited for preventing or treating seborrhoea and/or hirsutism and/or androgen-dependent alopecia.

Lysyl and/or Prolyl Hydroxylase-Inhibiting Anti-Hair Loss Active

Preferred examples of lysyl and/or propyl hydroxylase inhibitors which can be used according to the present invention are 2,4-diaminopyrimidine 3-oxide, or 2,4-DPO, which is described in Patent Application WO 96/09048, and 2,4-diamino-6-piperidinopyrimidine 3-oxide, or Minoxidil, which is described in U.S. Pat. No. 4,139,619 and U.S. Pat. No. 4,596,812.

These components are, for example, present in the composition according to the invention at a level of 0.001% to 5% by weight and, more preferably, at a level of 0.01% to 5% by weight, relative to the total weight of the composition.

The composition comprising the lysyl and/or prolyl hydroxylase inhibitor is advantageously used for preventing or treating alopecia.

Agent Stimulating the Synthesis of Dermal or Epidermal Macromolecules and/or Preventing their Degradation

Among the actives which stimulate the macromolecules of the dermis or prevent their degradation, mention may be made of those which act alternatively

on the synthesis of collagen, such as extracts of Centella asiatica; asiaticosides and derivatives; ascorbic acid or vitamin C and its derivatives; synthetic peptides such as iamin, the biopeptide CL or palmitoyloligopeptide sold by Sederma; peptides extracted from plants, such as the soya hydrolysate sold by Coletica under the trade name Phytokine®; and plant hormones such as auxins and lignans;

or on elastin synthesis, such as the extract of Saccharomyces cerevisiae sold by LSN under the trade name Cytovitin®; and the algal extract of Macrocystis pyrifera sold by Secma under the trade name Kelpadelie®;

or on the synthesis of glycosaminoglycans, such as the fermentation product of milk by Lactobacillus vulgaris, sold by Brooks under the trade name Biomin yogourth®; the extract of brown alga Padina pavonica sold by Alban Müller under the trade name HSP3®; and the extract of Saccharomyces cerevisiae available in particular from Silab under the trade name Firmalift® or from LSN under the trade name Cytovitin®;

or on fibronectin synthesis, such as the extract of the zooplankton Salina sold by Seporga under the. trade name GP4G®; the yeast extract available in particular from Alban Müller under the trade name Drieline®; and the palmitoyl pentapeptide sold by Sederma under the trade name Matrixil®;

or on the inhibition of metalloproteinases (MMPS) such as, more particularly, MMPs 1, 2, 3 and 9. Mention may be made of the following: retinoids and derivatives, oligopeptides and lipopeptides, lipoamino acids, the malt extract sold by Coletica under the trade name Collalift®; extracts of blueberry or of rosemary; lycopene; isoflavones, their derivatives or plant extracts containing them, especially soya extracts (sold, for example, by Ichimaru Pharcos under the trade name Flavosterone SB®), or of red clover, of flax, of kakkon or of sage;

or on the inhibition of serine proteases such as leukocyte elastase or cathepsin G. Mention may be made of the following: the peptide extract of Leguminosa seeds (Pisum sativum) sold by LSN under the trade name Parelastyl®; heparinoids; and pseudodipeptides such as {2-[acetyl-(3-trifluoromethylphenyl)amino]-3-methyl-butyrylamino}acetic acid.

Among the actives which stimulate epidermal macromolecules, such as fillagrin and keratins, mention may be made in particular of the extract of lupin sold by Silab under the trade name Structurine®; the extract of Fagus sylvatica beech buds sold by Gattefosse under the trade name Gatuline®; and the extract of the zooplankton Salina sold by Seporga under the trade name GP4G®.

The composition according to the invention comprising one or more of the above compounds is particularly suitable for use in the preventing or treatment of the cutaneous signs of ageing, in particular the loss of firmness and/or elasticity of the skin.

Agent Stimulating Fibroblast or Keratinocyte Proliferation and/or Keratinocyte Differentiation

Agents which stimulate fibroblast proliferation and can be used in the composition according to the invention may be selected, for example, from plant proteins or polypeptides, extracted in particular from soya (for example an extract of soya sold by LSN under the name Eleseryl SH-VEG 8® or sold by Silab under the trade name Raffermine®); and plant hormones such as gibberellins and cytokinins.

Agents which stimulate the proliferation of keratinocytes and can be used in the composition according to the invention include, in particular, retinoids such as retinol and its esters, including retinyl palmitate; adenosine; phloroglucinol; walnut cake extracts sold by Gattefosse; and extracts of Solanum tuberosum sold by Sederma.

Agents which stimulate keratinocyte differentiation include, for example, minerals such as calcium; the extract of lupin sold by Silab under the trade name Photopreventine®; the sodium beta-sitosteryl sulphate sold by Seporga under the trade name Phytocohesine®; and the maize extract sold by Solabia under the trade name Phytovityl®; and lignans such as secoisolariciresinol.

The composition according to the invention comprising these compounds is preferably intended for use for preventing or treating the cutaneous signs of ageing.

Muscle Relaxant or Dermal Decontractant

The muscle relaxants or dermal decontractants according to the invention include alverine and its salts, manganese gluconate, diazepam, Argireline hexapeptide R, sold by Lipotec, certain secondary and tertiary carbonyl amines, adenosine, and also sapogenins and natural extracts, in particular of wild yam, containing them, and also extracts of Boswellia serrata.

The free-radical scavengers which can be used in the composition according to the invention include vitamin E and its derivatives such as tocopheryl acetate; bioflavonoids; co-enzyme Q10 or ubiquinone; certain enzymes such as catalase, superoxide dismutase and wheatgerm extracts containing them, lactoperoxidase, glutathione peroxidase and quinone reductases; glutathione; benzylidenecamphor; benzylcyclanones; substituted naphthalenones; pidolates; phytantriol; gamma-oryzanol; guanosine; lignans; and melatonin.

Calmatives

Useful calmatives that may be used in the composition according to the invention include the following: pentacyclic triterpenes and plant extracts (e.g.: Glycyrrhiza glabra) containing them, such as β-glycyrrhetinic acid and its salts and/or its derivatives (glycyrrhetinic acid monoglucuronide, stearyl glycyrrhetinate, 3-stearoyloxyglycyrrhetic acid), ursolic acid and its salts, oleanolic acid and its salts, betulinic acid and its salts, an extract of Paeonia suffruticosa and/or lactiflora, salts of salicylic acid and especially zinc salicylate, phycosaccharides from Codif, an extract of Laminaria saccharina, canola oil, bisabolol and camomile extracts, allantoin, Sepivital EPC (phosphoric diester of vitamin E and C) from SEPPIC, omega-3 unsaturated oils such as musk rose oil, blackcurrant oil, echium oil, fish oil, plankton extracts, capryloylglycine, Seppicalm VG (sodium palmitoylproline and Nymphea alba) from SEPPIC, an extract of Pygeum, an extract of Boswellia serrata, an extract of Centipeda cunninghami, an extract of Helianthus annuus, an extract of Linum usitatissimum, tocotrienols, extracts of Cola nitida, piperonal, an extract of clove, an extract of Epilobium angustifolium, aloe vera, an extract of Bacopa monieri, phytosterols, cortisone, hydrocortisone, indomethacin and betamethasone.

Lipolytic Actives or Actives Having a Favourable, Direct or Indirect, Activity on Decreasing Adipose Tissue

The derivatives capable of promoting lipolysis include the following:

1) phosphodiesterase inhibitors, such as:

xanthene derivatives such as caffeine and its derivatives, particularly the 1-hydroxyalkylxanthines described in document FR-A-2,617,401, caffeine citrate, theophylline and its derivatives, theobromine, acefylline, aminophylline, chloroethyltheophylline, diprofylline, diniprophylline, etamiphylline and its derivatives, etofylline and proxyphylline;

combinations containing xanthene derivatives, such as the combination of caffeine and silanol (caffeine methyl silanetriol derivative) and, for example, the product sold by Exsymol under the name cafeisilane C;

compounds of natural origin containing xanthine bases, and particularly caffeine, such as extracts of tea, of coffee, of guarana, of mate, of cola (Cola nitida) and in particular the dry extract of guaraná fruit (Paulina sorbilis) containing 8% to 10% of caffeine;

ephedrine and its derivatives which may be found particularly in natural form in plants such as Ma Huang (Ephedra plant).

2) plant extracts and extracts of marine origin, which either are active on the receptors to be inhibited, such as the β-2-blockers, the NPY-blockers (described in Patent EP 838217), or which inhibit the synthesis of LDL or VLDL receptors, or are active in stimulating β-receptors and G proteins, leading to the activation of adenylcyclase;

3) peptides or proteins

the peptides derived from parathyroid hormone, as described in Patents FR 2 788058 and FR 2781231 from Sederma or the peptides described in document FR 2 786 693, or even any other peptide having lipolytic properties;

protamines and derivatives thereof such as those described in document FR-A-2,758,724.

Agents which Act on the Microcirculation

Useful actives acting on the microcirculation (vasoprotectors or vasodilators) include those selected from flavonoids, ruscogenins, esculosides, escin extracted from common horsechestnut, nicotinates, heperidine methyl chalcone, essential oils of lavender or of rosemary, and extracts of Ammi visnaga.

The amount of these actives may vary widely. Generally speaking, these actives are present at a concentrating ranging from 0.01% to 15% and preferably from 0.05% to 10% by weight relative to the total weight of the composition.

Agents which Act on the Energy Metabolism of Cells

The actives in question are those which act on the energy metabolism of skin, such as, for example, and without limitation, ATP synthesis, and also those which are involved in the respiratory chain of the cell or in the energy stores. Mention may be made of coenzyme Q10 (ubiquinone), cytochrome C, creatine or else phosphocreatine.

Actives particularly suitable for the implemtation of the invention are vitamins, in particular coenzyme Q10, vitamin B3 and vitamin C and derivatives thereof, ellagic acid and generally depigmenting agents, retinol and retinoids, myorelaxant or dermal decontractant, lipolytic actives or actives having a favourable activity on decreasing adipose tissue.

According to one of the advantageous embodiments of the invention, the octanol/water partition coefficient of the cosmetic active principle, expressed in terms of log P and calculated in a way familiar to the skilled person, may vary from −2 to +4.

The invention likewise provides a composition which comprises (i) a water-in-oil emulsion comprising a fatty phase with a polarity δa greater than or equal to 0.1 and comprising a mixture of at least two oils as defined in the present text, and (ii) a cosmetic or dermatological active, as a combination product for use conjointly, simultaneously, separately or spread out over time, by topical application. Said composition is in particular a combination product (kit of part) which may be in the form of a kit comprising formulations (i) and (ii), corresponding to the compositions defined above, which are provided to the user, who applies the two compositions simultaneously, separately or spread out over time. The kit may take the form of a container comprising at least one compartment, and in particular at least two compartments each containing, respectively, at least component (i) and at least component (ii).

The invention further provides for the use of a composition in the form of a water-in-oil emulsion comprising a fatty phase with a polarity δa greater than or equal to 0.1 and comprising a mixture of at least two oils, as defined above, for promoting the penetration of a cosmetic active and/or for preparing a composition intended for promoting the penetration of a dermatological active.

According to one embodiment the composition in W/O emulsion form will contain at least one surfactant selected from polyglycerol alkyl derivatives, alkylated polyethylene glycols, alkyl derivatives of sorbitan, metal salts of fatty acids, and polyolefin-derived oligomers or polymers; with preference it will contain at least one surfactant of polyolefin-derived oligomer or polymer type having an esterified succinic end group.

Said composition will be useful for promoting the penetration of actives defined above, and in particular actives such as muscle relaxants or dermal decontractants, depigmenting agents and/or agents stimulating the synthesis of dermal or epidermal macromolecules. Said composition and said cosmetic or dermatological active will be able to be applied conjointly, the active being in a mixture with the W/O composition, or simultaneously, with a time interval or sequentially over time.

In particular, the W/O composition defined above is a pretreatment composition.

Further advantages of the invention will emerge from a reading of the examples which follow.

### EXAMPLE 1

Skin Absorption Experiment

Formulations Tested

A number of water-in-oil emulsion type compositions are prepared which comprise a fatty phase composed of at least two oils having a particular δa. These compositions are expressed in terms of percentage by mass.

| | A | B | C | D |
|---|---|---|---|---|
| A - AMMONIUM | 0.2 | 0.2 | 0.2 | 0.2 |
| POLYACRYLOYLDIMETHYL | | | | |
| TAURATE | | | | |
| Salts | 2 | 2 | 2 | 2 |
| Preservatives | 0.2 | 0.2 | 0.2 | 0.2 |
| Water | qs 100 | qs 100 | qs 100 | qs 100 |
| B - POLYISOBUTYLENE | 1.92 | 1.92 | 1.92 | 1.92 |
| CONTAINING ESTERIFIED | | | | |
| SUCCINIC END GROUP* | | | | |
| DIMETHICONE | 0 | 1.78 | 1.77 | 0.21 |
| SQUALANE | 0 | 2.66 | 2.65 | 0.32 |
| ISONONYL ISONONANOATE | 0 | 3.56 | 3.54 | 0.43 |
| HYDROGENATED | | | 0 | 7 |
| POLYISOBUTENE | | | | |
| PRUNUS ARMENIACA | 15 | 7 | | |
| ZINC OXIDE | 0.5 | 0.5 | 0.5 | 0.5 |
| C-NYLON-12 | 3 | 3 | 3 | 3 |
| Polarity of fatty | 4.8 | 4.0 | 3.2 | 0.4 |
| phase (δa in J^{1/2} cm^{-3/2}) | | | | |

Procedure:

For phase A: homogenize the AMMONIUM POLYACRYLOYLDIMETHYL TAURATE in water and then mix it with all of the constituents of phase A at ambient temperature.

Mix and homogenize phase B.

Slowly add phase A to phase B to give the emulsion.

*commercial reference used: Lubrizol 5603 from Lubrizol.

Evaluation Protocol

The study is carried out on complete human skin. The skin samples are mounted in diffusion cells in static mode, of “In-Line” type, measuring 0.5 cm², for a total desorption period of 16 hours.

Formulations A, B, C and D are applied at a rate of 2 mg/cm² to the surface of the skin as a pretreatment. One hour following this application, a solution of active (caffeine radiolabelled with ¹⁴C) is applied. A series of control skin samples receives the solution of caffeine directly without pretreatment by the formulation.

Following application, the surface of the samples is washed. Only the analysis of the surface excesses is carried out by assaying the ¹⁴C caffeine. The amounts of [¹⁴C]caffeine and/or [¹⁴C]derivatives recovered in the surface excesses are expressed in terms of % of caffeine/dose applied.

Results

| % surface excess | A | B |
|---|---|---|
| with pretreatment | 62.53 | 42.94 |
| without pretreatment | 68.03 | 68.03 |
| % reduction in caffeine at the surface of | −8.08 | −36.88 |
| the skin relative to the sample without | | |
| pretreatment | | |

The comparison of the results obtained for the pretreatments with cosmetic compositions A and B shows that composition B, which contains a mixture of oil, relative to composition A, which contains only a single oil, permits a more substantial decrease in the active at the surface of the skin, corresponding to the improved dispersal of the caffeine at the level of the skin.

| % surface excess | C | D |
|---|---|---|
| with pretreatment | 46.67 | 61.97 |
| without pretreatment | 82.06 | 82.06 |
| % reduction in caffeine at the surface | −43.13 | −24.48 |
| of the skin relative to the sample | | |
| without pretreatment | | |

The comparison of the results obtained for the pretreatments with cosmetic compositions C and D shows that composition C, which contains an oily phase having a greater 6a than the oily phase of composition D, permits better skin passage of the active.

### EXAMPLE 2

Formulations E and F, of water-in-oil emulsion type, are prepared in accordance with the procedures described above.

| | E | F |
|---|---|---|
| AMMONIUM POLYACRYLOYL- | 0.2 | 0.2 |
| DIMETHYL TAURATE | | |
| Salts | 2 | 2 |
| Preservatives | qs | qs |
| POLYISOBUTYLENE | 1.92 | 1.92 |
| CONTAINING ESTERIFIED | | |
| SUCCINIC END GROUP* | | |
| NYLON-12 | 3 | 3 |
| DIMETHICONE | 6.01 | |
| ZINC OXIDE | 0.5 | 0.5 |
| SQUALANE | 8.99 | |
| ISONONYL ISONONANOATE | | 15 |
| Water | qs 100 | qs 100 |

The penetration of caffeine after pretreatment is evaluated in accordance with the protocol described in Example 1.

| % surface excess | E | F |
|---|---|---|
| with pretreatment | 60.87 | 83.85 |
| without pretreatment | 85.88 | 85.88 |
| % reduction in caffeine at the surface | −29.12 | −2.36 |
| of the skin relative to the sample | | |
| without pretreatment | | |

The comparison of the results obtained for the pretreatments with cosmetic compositions E and F shows that composition E, which contains a mixture of oil, relative to composition A, which contains only a single oil, permits a more substantial decrease in the active at the surface of the skin, corresponding to the improved dispersal of the caffeine at the level of the skin.

The above written description of the invention provides a manner and process of making and using it such that any person skilled in this art is enabled to make and use the same, this enablement being provided in particular for the subject matter of the appended claims, which make up a part of the original description and including a method of promoting the penetration of a cosmetic active, wherein a composition in the form of a water-in-oil emulsion is applied topically, said emulsion comprising a fatty phase with a polarity δa greater than or equal to 0.1 and comprising a mixture of at least two oils, and in that the application of said emulsion is made simultaneously, with an interval or sequentially in time with the application of the cosmetic active. Another preferred embodiment of the invention similarly fully described and enabled is a method of promoting the penetration of a cosmetic active according to any one of the preceding claims, wherein it comprises the following steps:

(a) the water-in-oil emulsion is applied to an area of the skin and/or scalp,

(b) the emulsion is left in contact with the skin and/or scalp for a time greater than or equal to 2 minutes,

(c) the cosmetic active is applied to the same area of the skin and/or scalp as that of step (a). Also enabled is a composition wherein it comprises (i) a water-in-oil emulsion comprising a fatty phase with a polarity δa greater than or equal to 0.1 and comprising a mixture of at least two oils as defined in one of the preceding claims, and (ii) a cosmetic or dermatological active, as a combination product for use conjointly, simultaneously, separately or spread out over time, by topical application.

As used above, the phrases “selected from the group consisting of,” “chosen from,” and the like include mixtures of the specified materials. Terms like “contain(s)” etc are open terms that mean including at least.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method of promoting the penetration of a cosmetic active, wherein a composition in the form of a water-in-oil emulsion is applied topically to keratin material simultaneously, with an interval, or sequentially in time with the application of the cosmetic active, said emulsion comprising a fatty phase with a polarity δa greater than or equal to 0.1 and comprising a mixture of at least two oils.

2. A method of promoting the penetration of a cosmetic active according to claim 1, wherein the fatty phase of the emulsion is a mixture of at least three oils whose final polarity is greater than 0.1.

3. A method according to claim 1, wherein the polarity of the fatty phase of the emulsion is greater than or equal to 0.4.

4. A method according to claim 1, wherein the fatty phase of the emulsion comprises a mixture of apolar oils and polar oils.

5. A method according to claim 1, wherein the fatty phase of the emulsion comprises from 20% to 80% (w/w) of polar oils possessing δa values of between 2 and 11.

6. A method according to claim 5, wherein the individual polarities of the polar oils present in the fatty phase correspond to 8a values of 3 to 8.

7. A method according to claim 5, wherein the fatty phase comprises from 40% to 75% of polar oils.

8. A method according to claim 1, wherein the fatty phase of the emulsion has a polarity δa of less than or equal to 8.

9. A method according to claim 1, wherein the emulsion comprises at least one polar oil selected from silicone oils, vegetable oils having a high triglyceride content, and synthetic oils including synthetic esters.

10. A method according to claim 9, wherein the emulsion comprises at least one oil selected from dimethicones, isononyl isononanoate, isopropyl lauroyl sarcosinate and apricot kernel oil.

11. A method according to claim 1, wherein the amount of the fatty phase in the emulsion is from 0.01% to 50% by weight relative to the total weight of the composition.

12. A method of promoting the penetration of a cosmetic active according to claim 1, further comprising the following steps:
(a) the water-in-oil emulsion is applied to an area of the skin and/or scalp,
(b) the emulsion is left in contact with the skin and/or scalp for a time greater than or equal to 2 minutes,
(c) the cosmetic active is applied to the same area of the skin and/or scalp as that of step (a).

13. A method according to claim 12, wherein steps (a) and (c) are carried out simultaneously and in that the cosmetic active is present in the composition in the form of a water-in-oil emulsion.

14. A method according to claim 12, wherein steps (a) and (c) are carried out sequentially in time and in that the cosmetic active is present in a cosmetic composition comprising a physiologically acceptable medium.

15. A method according to claim 1, wherein the cosmetic active is selected from moisturizers, desquamating agents, anti-seborrhoeic agents, anti-ageing agents, depigmenting agents, pro-pigmenting agents, muscle relaxants or dermal decontractants, calmatives, lipolytic or slimming agents, microcirculation promoters and anti-hair-loss actives.

16. A method according to claim 1, wherein the water-in-oil emulsion further comprises at least one surfactant selected from polyglycerol alkyl derivatives, alkylated polyethylene glycols, alkyl derivatives of sorbitan, metal salts of fatty acids, silicone surfactants and polyolefin-derived oligomers and polymers.

17. A method according to claim 16, wherein the water-in-oil emulsion comprises at least one surfactant of polyolefin-derived oligomer or polymer type having an esterified succinic end group.

18. A composition comprising (i) a water-in-oil emulsion comprising a fatty phase with a polarity δa greater than or equal to 0.1 and at least two oils, and (ii) at least one cosmetic or dermatological active.

19. The composition according to claim 18, wherein it further comprises at least one surfactant of polyolefin-derived oligomer or polymer type having an esterified succinic end group.
